(19) United States
(12) Patent Application Publication
(10) Pub. No.: US 2016/0374776 A1
(43) Pub. Date: Dec. 29, 2016

(54) MOBILE DEVICE PATIENT IDENTIFIER

(71) Applicant: Dental Imaging Technologies Corporation, Hatfield, PA (US)

(72) Inventors: Darryl Day Spencer, Furlong, PA (US); George John Cocco, Havertown, PA (US); Michael Joseph Parma, Chalfont, PA (US); Bradley S. Carlson, Doylestown, PA (US)

(21) Appl. No.: 14/752,460

(22) Filed: Jun. 26, 2015

(51) Int. Cl. A61B 90/96; A61B 5/00

(52) U.S. Cl. CPC A61B 90/96; A61B 5/7435; G06Q 50/22

(57) ABSTRACT

A method and system is disclosed for using a computer-readable, graphical code on a mobile device to deliver a medical service. The method can include accessing appointment data via a mobile software application and checking-in to the appointment and to medical service rooms by using the mobile software application. In one embodiment, the mobile software application includes a computer-readable, graphical code, and in another construction, the mobile software application includes the use of a micro-location device. Other methods, and systems are also disclosed.

### BACKGROUND

Embodiments of the invention relate to methods of using an electronic patient identifier to check in at a medical facility and to link the patient identifier with medical images, image acquisition devices, and medical information systems.

### SUMMARY

In one embodiment, the invention provides a method of using a computer-readable, graphical code on a mobile device to deliver a medical service. The method includes requesting a medical service for a patient, generating an appointment to perform the medical service for the patient, and generating a computer-readable, graphical code patient identifier. The patient identifier is linked to appointment information for the appointment and patient information in a data storage device. The patient identifier including the appointment information and the patient information is sent to a mobile device of the patient. The patient brings the mobile device with the patient identifier to the medical facility. At the medical facility, a terminal reads, by, for example, an optical scanner, the patient identifier. When the patient identifier matches the appointment information and patient information for the patient stored in the data storage device, the patient is admitted.

In another embodiment, the invention provides a method of using an electronic micro-location patient identifier on a mobile device to deliver a medical service. The method includes requesting a medical service for a patient, generating an appointment to perform the medical service for the patient, and generating an electronic micro-location patient identifier. The patient identifier is linked to appointment information for the appointment and patient information in a data storage device based on the request for the medical service. The patient identifier including the appointment information and the patient information is sent to a mobile device. The patient identifier is then transmitted, by the mobile device to a micro-location device configured to check-in patients. Transmission of the patient identifier may occur, for example, at a reception area in a medical facility when the mobile device comes within a transmission range of the micro-location device. The method includes checking-in the patient at the medical facility to perform the medical service during an appointment window when the electronic micro-location patient identifier matches the appointment information and patient information for the patient stored in the data storage device.

In another embodiment, the invention provides a system configured to use an electronic patient identifier on a mobile device for medical services. The system includes a mobile device to receive an electronic patient identifier associated with a patient and a computer that generates appointment information for an appointment to perform a medical service for the patient. The computer generates a computer-readable, graphical code patient identifier linked to the appointment information and patient information. The computer sends the patient identifier including the appointment information and the patient information to the mobile device. The patient is admitted at a medical facility near a time of the appointment by reading the patient identifier with a scanner. Additionally, a data storage device at the medical facility stores the appointment information, the patient information, and the electronic patient identifier associated with the patient.

Other aspects of the invention will become apparent by consideration of the detailed description and accompanying drawings.

### BRIEF DESCRIPTION OF THE DRAWINGS

FIG. 1 schematically illustrates a workflow for an imaging process where a patient is initially at a location remote from a medical facility (e.g., the patient's home) and continuing through areas within the facility including a reception area, an examination or consultation room, and continuing to a room housing an imaging device.

FIG. 2 illustrates a graphical user interface (“GUI”) on a mobile device of a patient pass application, where the GUI includes a computer-readable, graphical code such as QR code, a barcode, or the like.

FIG. 3 illustrates a GUI on a mobile device of a patient pass application including a computer-readable, graphical code, text information, and updated insurance information.

FIG. 4 illustrates a workflow where the patient has arrived at the reception area of the medical facility and a computer-readable, graphical code displayed in the GUI of the patient pass application.

FIG. 5 illustrates a patient in a reception area of a medical facility where the patient uses a mobile device to interact with a reader or similar device in the reception area and, in the particular example illustrated, an optical reader reads the computer-readable, graphical code displayed in the GUI of the patient pass application.

FIG. 6 illustrates a link that occurs between patient information metadata and medical facility metadata in a data storage device when the patient checks-in with the computer-readable, graphical code.

FIG. 7 illustrates the GUI of the patient pass application including updated insurance information and a checked-in confirmation message and status indicator.

FIG. 8 illustrates a workflow where the patient has arrived at the consultation room of the medical facility and GUI of the patient pass application includes a computer-readable, graphical code.

FIG. 9 illustrates a workflow where the GUI of the patient pass application or a computer-readable, graphical code of the medical professional is scanned in the consultation MOM.

FIG. 10 illustrates a GUI for imaging software running on a computer in the consultation room of the medical facility showing patient, medical professional, and specified device information associated with subsequently acquired images.

FIG. 11 illustrates a link that occurs between patient information metadata, medical facility metadata, medical professional metadata, and specified device metadata stored on an image or the data storage device when the patient is located in the consultation room.

FIG. 12 illustrates a workflow where a device located within the consultation room and having a GUI with a computer-readable, graphic code that is scanned or read by a device operated by a medical professional.

FIG. 13 illustrates a workflow where the patient interacts with a physician or other medical professional and the computer-readable, graphical code of the GUI of the patient pass application or a computer-readable, graphical code of the medical professional is scanned in the imaging room of the medical facility by a device carried by the medical professional or, alternatively, by a device located within the imaging room.

FIG. 14 illustrates a workflow where a computer-readable, graphic code of a device located within the imaging room is scanned by a device operated by a second medical professional.

FIG. 15 illustrates the GUI for imaging software running on a computer in the imaging room of the medical facility showing patient, medical professional, and imaging device information associated with subsequently acquired images.

FIG. 16 illustrates a link that occurs between patient information metadata, medical facility metadata, second medical professional metadata, and second specified device metadata stored on an image or the data storage device when the patient is located in the imaging room.

FIG. 17 schematically illustrates a workflow for an imaging process where a patient is initially at a location remote from a medical facility (e.g., the patient's home) and continuing through areas within the facility including a reception area, an examination or consultation room, and continuing to a room housing an imaging device.

FIG. 18 illustrates a GUI on a mobile device of a patient pass application, where the GUI includes a micro-location indicator.

FIG. 19 illustrates the workflow where the GUI of the patient pass includes a micro-location indicator indicating that the micro-location device is operational, and text information that includes a patient name, a patient identifier, a medical practice or facility name, and an appointment date.

FIG. 20 illustrates a graphical user interface on a mobile device of a patient pass including updated insurance information, micro-location indicator, and checked-in confirmation message.

FIG. 21 illustrates a link that occurs between patient information metadata and medical facility metadata in a data storage device when the patient checks-in with the micro-location device.

FIG. 22 illustrates a workflow where the patient has arrived at the consultation room of the medical facility and the mobile device includes a micro-location device that transmits a patient pass via the patient pass application.

FIG. 23 illustrates a workflow where the patient interacts with a physician or other medical professional and the micro-location device and the patient pass application of the mobile device communicate with a micro-location device of a device carried by the medical professional or, alternatively, by micro-location device of a device located within the consultation room.

FIG. 24 illustrates a GUI for imaging software running on a computer in the consultation room of the medical facility showing patient, medical professional, and imaging device information associated with subsequently acquired images.

FIG. 25 illustrates a link that occurs between patient information metadata, medical facility metadata, medical professional metadata, and imaging device metadata stored on an image or the data storage device when the patient is located in the consultation room.

FIG. 26 illustrates a workflow where the patient interacts with a physician or other medical professional and the micro-location device communicates with a micro-location device carried by the medical professional or, alternatively, by a micro-location device located within the imaging room.

FIG. 27 illustrates a workflow where a micro-location device located within the imaging room communicates with a micro-location device operated by a second medical professional and the micro-location device for the patient pass application.

FIG. 28 illustrates a GUI for imaging software running on a device in the imaging room of the medical facility showing patient, medical professional, and imaging device information associated with subsequently acquired images.

FIG. 29 illustrates a link that occurs between patient information metadata, medical facility metadata, medical professional metadata, and imaging device metadata stored on an image or in the data storage device when the patient is located in the imaging room.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways.

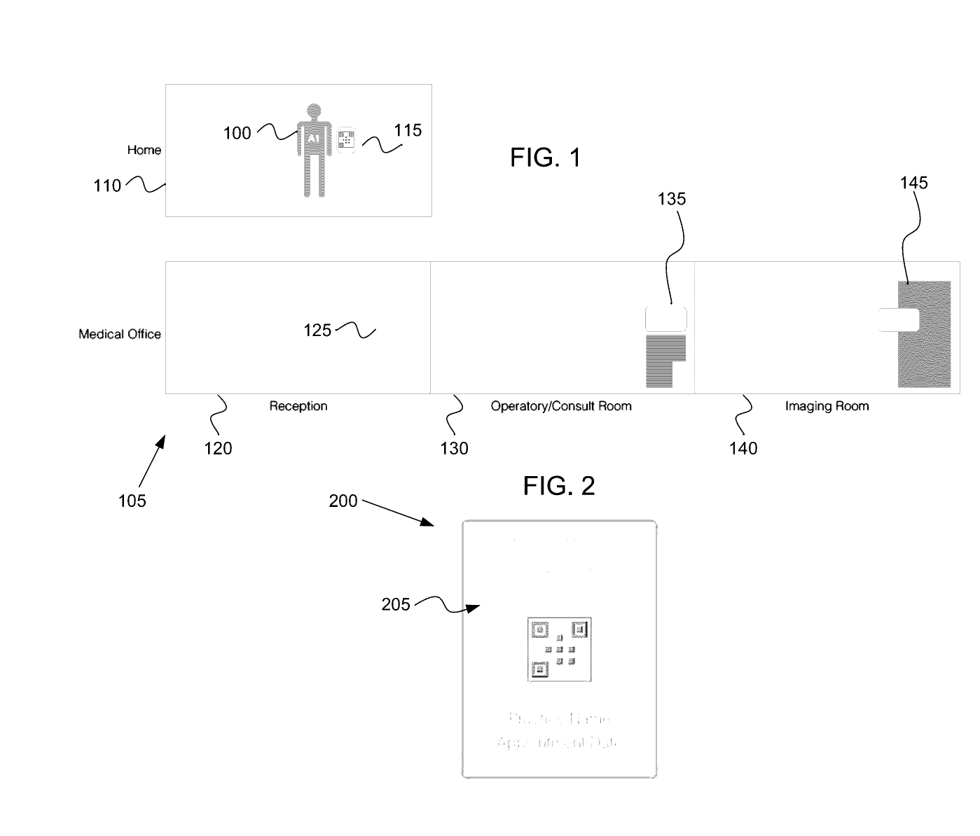

FIG. 1 illustrates a workflow diagram of an exemplary process of a patient 100 visiting a medical facility 105 for a medical service. The medical facility 105 may include, for example, a hospital, a clinic, a dental office, a physician's office, or a mobile treatment center. Before coming to the medical facility 105, the patient 100 is initially at a location remote from a medical facility (e.g., the patient's home 110). The patient 100 has a mobile device 115, such as, for example, a smart phone, a tablet, or a laptop computer. The medical facility 105 includes a plurality of rooms that are designed to perform different functions and to house different diagnostic equipment. The workflow proceeds through one or more rooms within the medical facility 105. A reception area 120 is where the patient 100 checks-in for the medical service. The reception area 120 includes a reception area terminal 125. The reception area terminal 125 may be configured to enable the patient 100 to check-in to the medical facility without the need to interact with a receptionist, office assistant, or other staff member.

A consultation room 130 is connected to the reception area 120. The consultation room 130 contains diagnostic equipment 135 and provides a private working space for performing the medical service. An imaging room 140 is connected to the consultation room 130. The imaging room 140 contains additional equipment for diagnostic testing including at least one imaging device 145.

Prior to the appointment at the medical facility 105, the patient installs a software application in the form of a patient pass 200 on the mobile device 115. As shown in FIG. 2, the patient pass 200, when run, generates a graphical user interface (GUI 205). The GUI 205 provides information to the patient 100 and provides the patient 100 the ability to input new or updated information into the patient pass 200. The device 115 wirelessly communicates with a device at the medical facility 105 that sends out appointment information. The patient pass 200 is configured to receive the appointment information. Before the appointment time, an appointment acknowledgement message is sent to the mobile device 115 and processed by the patient pass 200. The GUI 205 displays the information about the upcoming appointment to the patient 100.

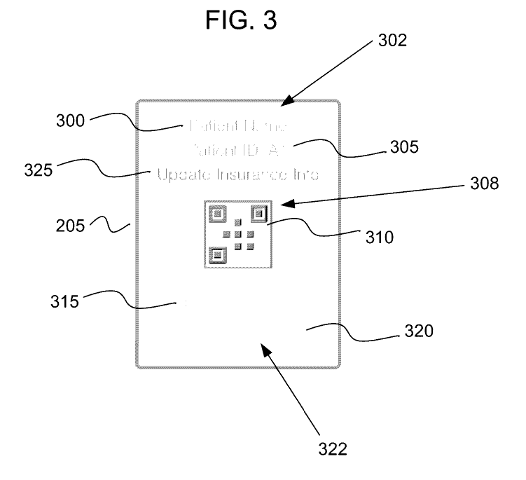

FIG. 3 illustrates information that is displayed on the GUI 205 of the patient pass 200. The patient name 300 is located at a top 302 of the GUI 205. Located under the patient name 300 is a patient identification number (i.e., patient ID 305). The patient ID 305 provides a unique identifier to identify the patient 100 and to track information associated with the patient 100 by the medical facility 105. In a center 308 of the GUI 205 is a graphical code 310 containing information about the patient 100. The information about the patient 100 includes, for example, a patient name 300, a patient ID 305, an update insurance link 325, a medical facility's name 315, and an appointment date 320. The graphical code 310 can include, for example, a two-dimensional (2D) barcode with various geometric patterns, a matrix barcode, a quick response (QR) code, an Aztec code, a data matrix code, a one-dimensional (1D) barcode, or other graphical codes. Located at a bottom 322 of the GUI 205 is the medical facility's name 315. Located under the medical facility's name 315 and above a bottom of the GUI 205 is the appointment date 320. The GUI 205 is not limited to the information as displayed or to the layout of the information.

The information in the patient pass 200 can be updated by the patient 100 through the GUI 205. For example, the GUI 205 includes the update insurance link 325 to allow the patient 100 to update the insurance information of the patient 100. In some embodiments, the patient pass 200 allows the patient 100 to update additional information on the patient pass 200 including the patient name 300, the patient ID 305, and the appointment date 320. The update insurance link 325, when activated, allows the patient 100 to enter new insurance information into the patient pass 200. The patient pass 200 saves the new insurance information into a mobile device memory of the mobile device 115. The patient pass 200 is configured to convert the new insurance information and generate a new or updated graphical code 310.

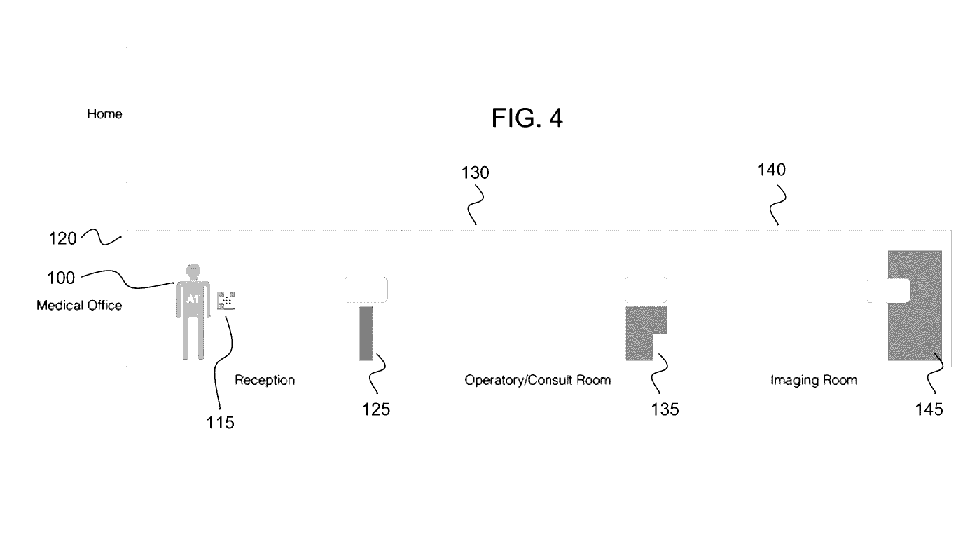

On the scheduled appointment date 320, the patient 100 arrives at the reception area 120 of the medical facility 105 as shown in FIG. 4. The patient 100 brings the mobile device 115 to the medical facility 105 and uses the patient pass 200 application on the mobile device 115 at various times during the visit at the medical facility 105. When the patient arrives, he or she activates the patient pass 200 on the mobile device 115. The patient pass 200 is configured to display the GUI 205, which includes the graphical code 310.

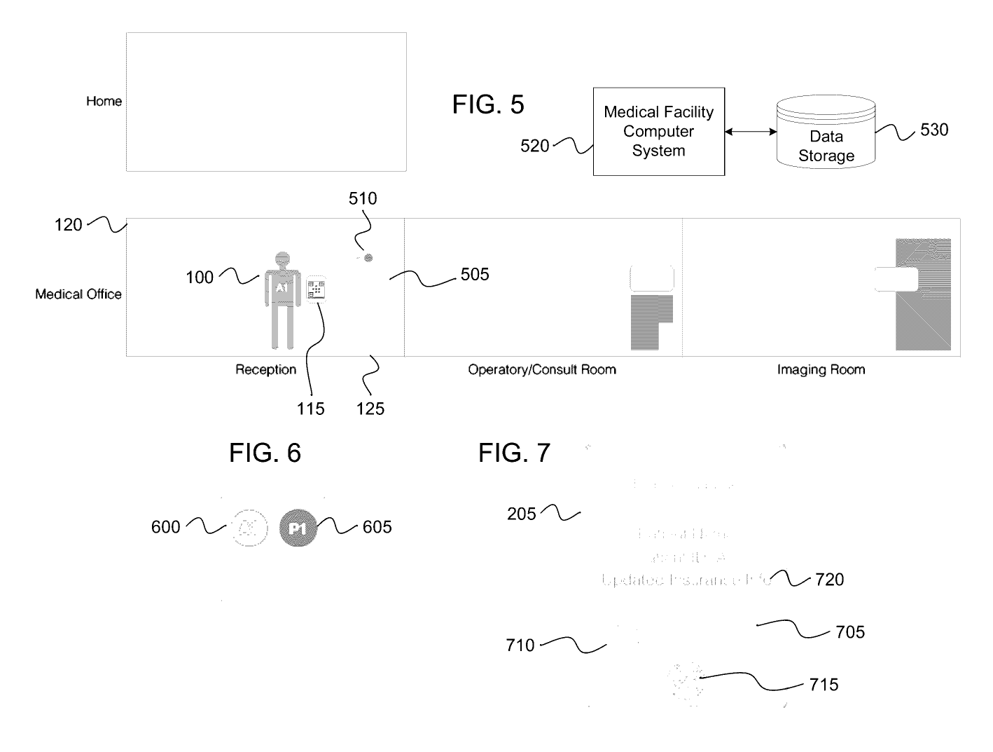

FIG. 5 illustrates a check-in process at the medical facility 105. The patient 100 checks-in by using the patient pass 200 and the reception area terminal 125 in the reception area 120. The reception area terminal 125 includes a user interface 505 and an optical scanner 510. The optical scanner 510 is adapted to read the graphical code 310 on the GUI 205. As described above, the graphical code 310 contains information about the patient 100, which includes the time of the appointment and the appointment date 320. When the patient 100 arrives at the medical facility 105 for the appointment, the patient 100 holds the mobile device 115 with the GUI 205 towards the optical scanner 510. The optical scanner 510 reads the graphical code 310, and using the information contained within the code, checks-in the patient 100. The optical scanner 510 is connected to a medical facility computer system 520 that includes a data storage device 530. The computer system 520 generates a notification for the staff of the medical facility to indicate that the patient 100 has checked-in.

It should be noted that the check-in process is not limited to a patient-operated check-in process. In other examples, a staff member of the medical facility 105 performs the patient check-in using the optical scanner 510 and the GUI 205 on the mobile device 115. In this example, the optical scanner 510 does not require a dedicated terminal. Instead, the optical scanner 510 may be a stand-alone device or portable device and connected (for example, wirelessly) to the medical facility computer system 520. In this example, the staff member checks-in the patient 100 using the optical scanner 510.

The data storage device 530 stores records and information relating to the patient 100. The reception area terminal 125 may be connected to the medical facility computer system 520 via a wired or a wireless connection (e.g., Wi-Fi, Bluetooth, etc.). This connection allows the medical facility computer system 520 to receive patient information from the reception area terminal 125. The medical facility computer system 520 can automatically update the data storage device 530 with the patient-entered insurance information. In other examples, the medical facility computer system 520 sends an alert to the office assistant to verify new patient information. The medical facility computer system 520 then updates the data storage device 530 with the updated patient information stored in the mobile device 115.

As illustrated in FIG. 6, the reception area terminal 125 links patient information 600, which is provided by the patient pass 200 to medical facility information 605, which is provided by the medical facility computer system 520. The medical facility information 605 includes, for example, the medical facility's name, the address of the medical facility 120, and the contact information of the medical facility 120. The patient information 600 and the medical facility information 605 are stored in the medical facility computer system 520 for later retrieval.

When the patient 100 has successfully checked-in, the medical facility computer system 520 wirelessly sends a check-in confirmation to the mobile device 115. The patient pass 200 receives the check-in confirmation and then displays a check-in confirmation message on the GUI 205 as illustrated in FIG. 7. In addition to the previous displayed information, the GUI 205 displays, for example, a “Barcode Confirmed” message 705 and a “Patient checked-In” message 710. Also, if the patient had updated the patient information on the patient pass 200, the patient pass 200 is configured to display a confirmation message on the GUI 205. For example, if the insurance information of the patient was changed, the GUI 205 displays an “Updated Insurance Info” message 720. The patient pass 200 also displays a status indicator such as, for example, a checkmark 715 indicating that the patient 100 has successfully checked-in. The GUI 205 also continues to display the medical facility's name 315, the patient name 300, and the patient ID 305.

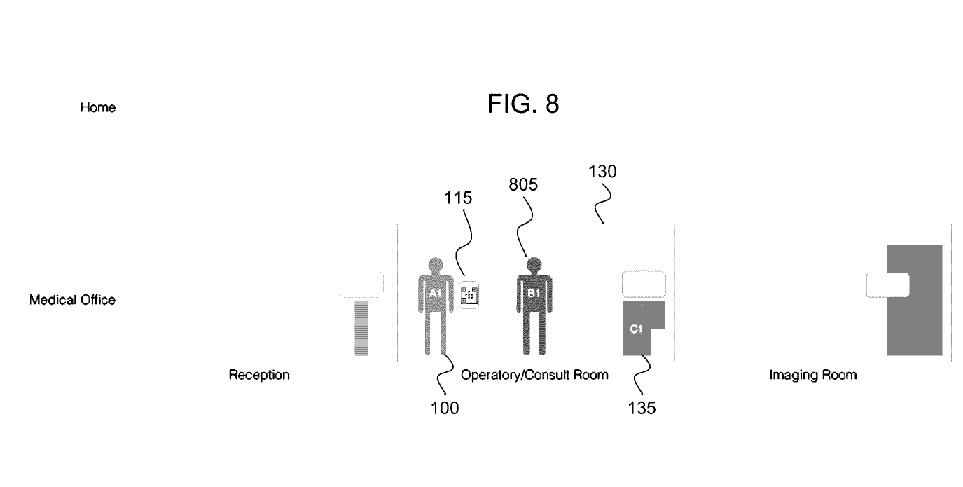

At the appointment time or once the medical professionals at the medical facility 105 are ready to see the patient 100 for the medical service, the patient 100 is directed to the consultation room 130, as shown in FIG. 8. In the consultation room 130, the patient 100 meets with a medical professional 805 for the requested medical service. As noted, the medical service may require diagnostic imaging. The medical professional 805 is trained to operate the imaging device 135. Before the medical professional 805 performs the diagnostic imaging, the medical professional 805 reads the patient information displayed on the GUI 205 of the patient pass 200.

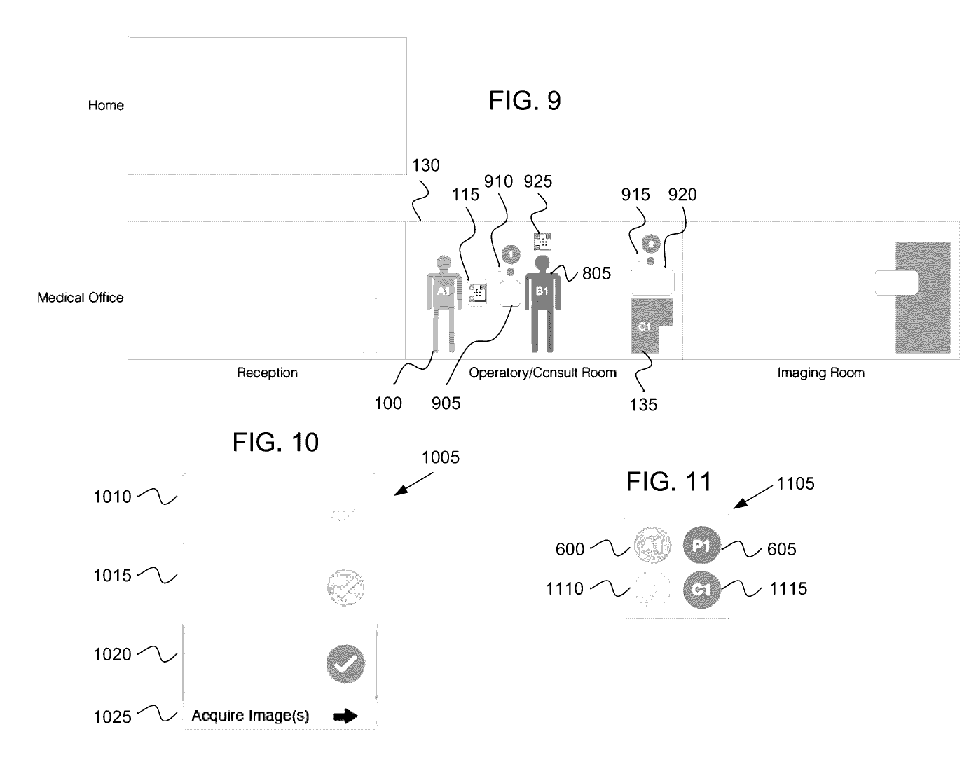

FIG. 9 illustrates two exemplary processes (Process A and Process B) for identifying, verifying, and recording the information provided by the patient pass 200. In both processes, the medical professional 805 has a caregiver code 925 (i.e., a computer-readable, graphical code), which uniquely identifies the medical professional 805. The caregiver code 925 may be displayed on a personal mobile device of the medical professional 805. Alternatively, the caregiver code 925 may be displayed on an identification badge. The medical professional 805 logs-in to a mobile device 910 or a module 920 with the caregiver code 925. If the medical professional 805 does not have the caregiver code 925, the medical professional 805 may log-in with a username and password unique to the medical professional 805.

After logging in, the medical professional 805 performs Process A or Process B. In Process A, the medical professional 805 uses the mobile device 905 having an optical scanner 910 to read the information about the patient 100 through the optical code 310. The medical professional 805 and the patient 100 simply hold their respective mobile devices, mobile device 115 and mobile device 905, next to each other, such that the graphical code 310 is read by the (caregiver's) mobile device 905. The information is entered into the mobile device 905. The mobile device transmits the information to the medical facility computer system 520. The medical professional 805 may use a mobile device 905 specifically assigned to him or her, in which case, the medical professional 805 carries the mobile device 905 from place to place or room to room. Alternatively, the mobile device 905 is kept within the consultation room 130 and is commonly used by one of more medical personnel.

In Process B, the medical professional 805 uses an optical scanner 915 located proximal to the imaging device 135, rather than on the mobile device 905. The optical scanner 915 is connected to the module 920. The module 920 includes interface software, which is adapted to receive patient information from the patient pass 200 via the graphical code 310. The module 920 and the imaging device 135 are connected to one another such that data may be transmitted between the devices. In one construction, the module can be separate device than the imaging device 135. In this construction, interface software is loaded separately onto the module 920 and configured to communicate with imaging software internal to the imaging device 135. In this way, information is shared between the imaging device 135 and the interface software on the module 920 so that images from the imaging device 135 can be linked with the information from the module 920. In another construction, the interface software on the module 920 may be included with or be a part of the imaging software on the imaging device 135.

Interface software on the mobile device 905 or alternatively on the module 920 includes a GUI 1005, as shown in FIG. 10. The GUI 1005 displays information to the medical professional 805 and to the patient 100. The GUI 1005 includes a “Personnel Name,” a “Personnel ID” 1010, a “Patient Name” and “Patient ID” 1015, and a “Device ID” 1020 for the imaging device 135. Checkmarks on the GUI 1005 associated with the currently displayed information indicate that information has been successfully recorded by the interface software. The GUI 1005 also displays an “Acquire Image(s)” link 1025 that, when pressed, tells the interface software to load an image from the imaging device 135. The medical professional 805 selects the Acquire Image(s) link 1025 after the medical professional 805 is satisfied with the diagnostic image that has been captured by the imaging device 135. After acquiring an image, the interface software links the diagnostic images with information that has been previously loaded into the interface software regarding that medical service.

Metadata 1105 is illustrated in FIG. 11. After acquiring an image, the GUI 1005 creates a link between patient metadata 600, medical facility metadata 605, medical professional metadata 1110, and imaging device metadata 1115. The interface software appends the metadata 1105 to the diagnostic image. The interface software communicates with the medical facility computer system 520. In one construction, the interface software communicates with the medical facility computer system 520 by a wired connection. In other constructions, the interface software can communicate wirelessly via near-field communications (e.g., Wi-Fi, Bluetooth, etc.). After the interface software collects the diagnostic image and the metadata 1105, the interface software transmits the combined information to the medical facility computer system 520. When the diagnostic images are retrieved from the facility computer system 520 they include the metadata 1105.

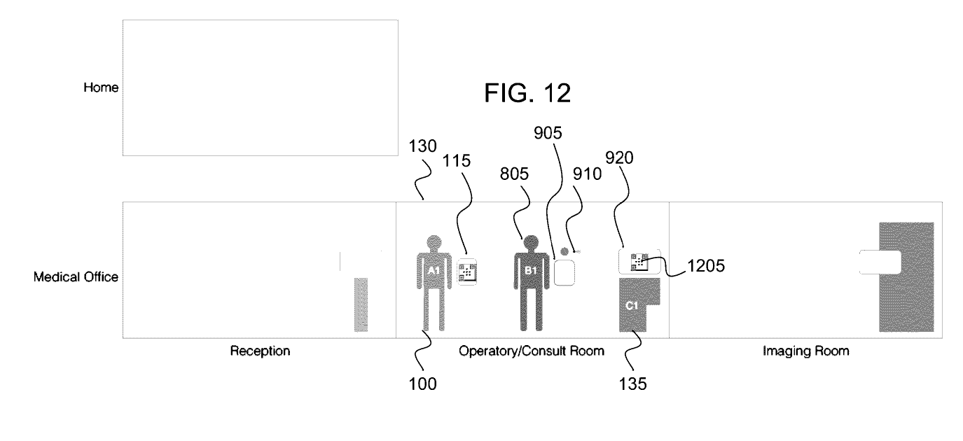

In another example, as illustrated by FIG. 12, the imaging device 135 includes an imaging device code 1205 (e.g., a computer-readable, graphical code). The imaging device code 1205 specifically identifies the imaging device 135 by an identification number. In the example shown, the imaging device code 1205 is displayed on the module 920. The imaging device code 1205 can be located on a display of the module 920. Alternatively, like the caregiver code 925, the imaging device code 1205 can be displayed on an identification tag located on the imaging device 135. The medical professional 805 scans the imaging device code 1205 with the mobile optical scanner 910 located on the mobile device 905. This allows the interface software to capture a unique identifier of the imaging device 135. The unique identifier is included in the imaging device metadata 1115.

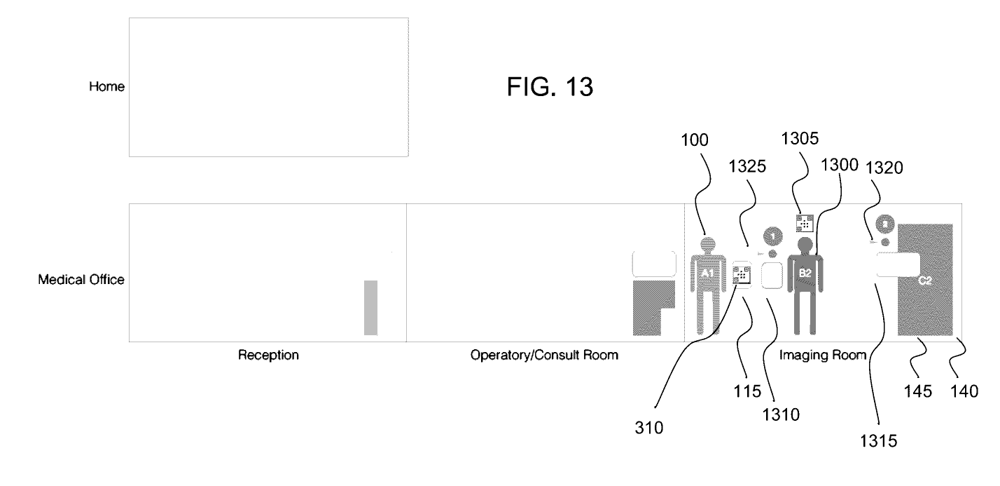

After evaluation in the consultation room 130, the patient 100 may require additional services such as testing or additional imaging, as shown in FIG. 13. The workflow in the imaging room 140 is similar to the workflow in the consultation room 130. The patient 100 meets with a medical professional 1300 on arrival in the imaging room 140. The medical professional 1300 has a graphical code 1305 (i.e., a computer-readable, graphical code), which uniquely identifies the medical professional 1300. The graphical code 1305 may be displayed on a personal mobile device of the medical professional 1300. Alternatively, the graphical code 1305 may be displayed on an identification badge. As in the previous examples, if the medical professional 1300 does not have the graphical code 1305, the medical professional 1300 may log in to the interface software. After the medical professional 1300 is identified in the interface software, the medical professional 1300 asks the patient 100 to present the patient pass 200. In one exemplary process (Process C), the medical professional 1300 uses a mobile device 1310 with an optical scanner 1325. In another exemplary process (Process D), the medical professional 1300 uses an imaging device 145 with an attached module 1315. In process D, the module 1315 includes an optical scanner 1320 and the interface software. Optionally, the imaging device 145 in the imaging room 140 can be the same kind of device as the diagnostic device 135 in the operatory/consult room 130, or can be a different kind of device. Examples of imaging devices include, but are not limited to intraoral or extraoral cameras, scanners or x-ray imaging devices such as intraoral x-ray unit, handheld x-ray unit, intraoral sensor, intraoral phosphor plate reader, panoramic x-ray unit or cone beam CT unit.

In Process C, the medical professional 1300 accesses the interface software on the mobile device 1310. Using the mobile device 1310, the medical professional 1300 scans the graphical code 310 on the mobile device 115 of the patient 100 with an optical scanner 1325. A link is created in the interface software between the medical professional 1300 and the patient 100.

In Process D, the medical professional accesses the interface software on the module 1315. An optical scanner 1320 is connected to the module 1315. The medical professional 1300 accesses the interface software by presenting an ID badge with the graphical code 1305 to the optical scanner 1320. The module 1315 is adapted to receive patent information from the optical scanner 1320. The module 1315 can be a separate device from the imaging device 145 or it can be a part of the imaging device 145. If the module 1315 is part of the imaging device 145, the interface software is part of the imaging software on the imaging device 145.

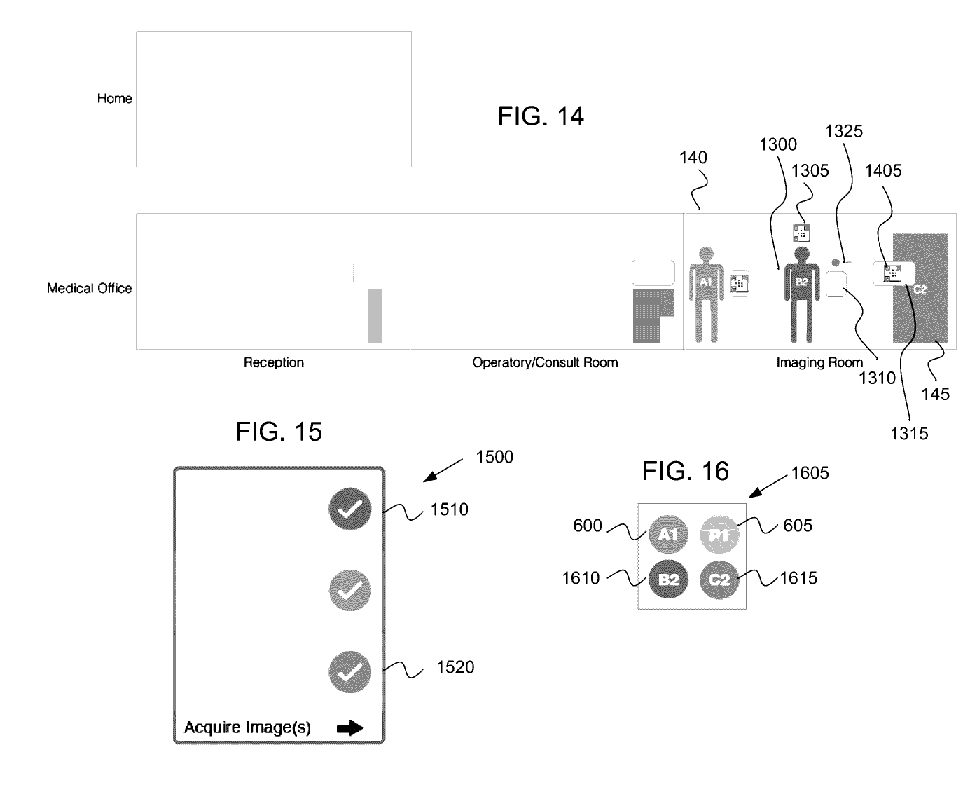

FIG. 14 illustrates a second part of Process C where the medical professional 1300 obtains information for the interface software from the imaging device 145. The imaging device 145 includes a device identification tag including a graphical code 1405 (i.e., a computer-readable, graphical code). The medical professional 1300 uses the optical scanner 1325 to read the graphical code 1405. The graphical code 1405 displays device information including, for example, a device type and a model number. The information about the imaging device 145 is loaded into the interface software.

FIG. 15 illustrates a GUI 1500 for the interface software. The GUI 1500 displays the newly collected information, for example, the GUI 1500 displays medical professional information 1510, patient information 1515, and device information 1520. The GUI 1500 displays the information so that the medical professional 1300 or the patient 100 can quickly determine if the collected information in the interface software is correct (e.g., that the medical professional 1300, the patient 100, and the imaging device 145 are correct).

Metadata 1605 collected by the interface software is illustrated in FIG. 16. The metadata 1605 links the patient metadata 600, the medical facility metadata 605, with the medical professional metadata 1610 from the medical professional 1300, and the imaging device metadata 1615 from the imaging device 145. The metadata 1605 is appended to the diagnostic images taken with the imaging device 145 and sent to the medical facility computer system 520.

In another embodiment, which is shown in FIG. 17, a short-range, wireless transceiver is utilized. Such a transceiver may operate in a radio-frequency range and may be referred to as a “micro-location” transceiver. Among other things, the micro-location transceiver is used to assist in the logistics of the medical service. A patient 1700 is at a location away from a medical facility 1705 (e.g., at the patient's home 1710). The medical facility computer system 520 sends a confirmation message of an upcoming appointment to a mobile device 1715 of the patient 1700. When the confirmation message is received, the mobile device 1715 alerts the patient 1700. The mobile device 1715 includes micro-location technology. For example, the mobile device 1715 can be equipped with the micro-location transceiver and be configured to wirelessly communicate with one or more micro-locations within the medical facility 1705.

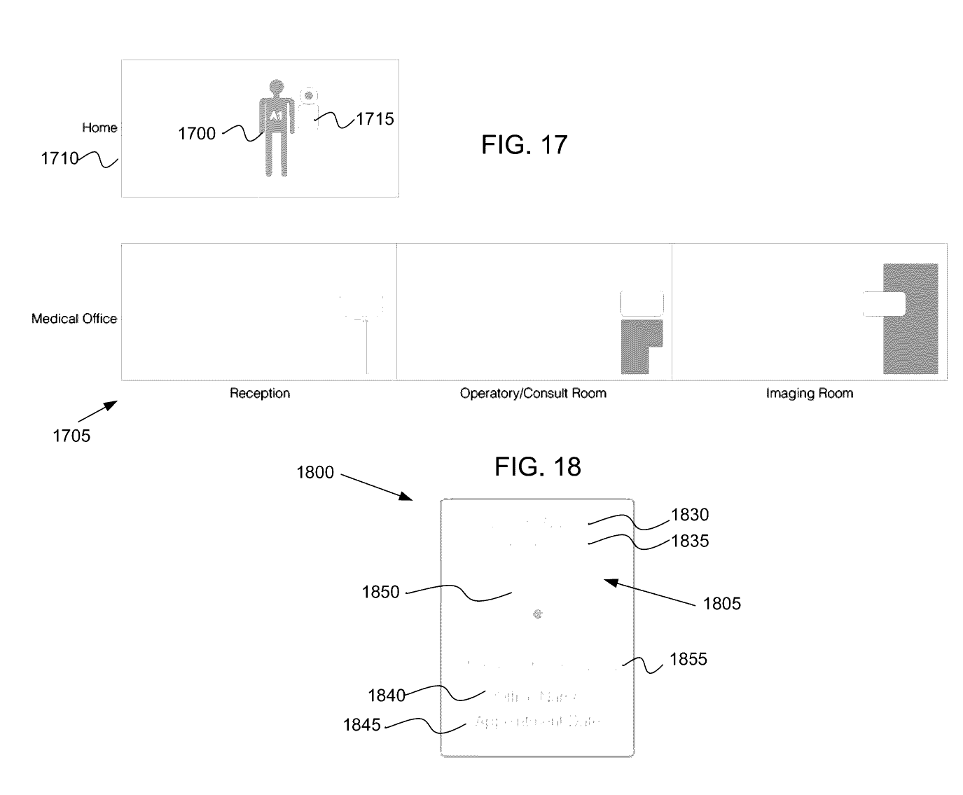

FIG. 18 illustrates a GUI 1805 of a patient pass 1800. In the embodiment shown, the GUI 1805 presents much of the same information as the GUI 205. The GUI 1805 displays a patient name 1830, a patient ID 1835, a medical facility's name 1840, and an appointment date 1845. Micro-location technology does not require the graphical code 310 as in the previous examples. Instead, the GUI 1805 displays a symbol 1850 indicating that micro-location is enabled. In addition, the GUI 1805 displays a micro-location enabled message 1855.

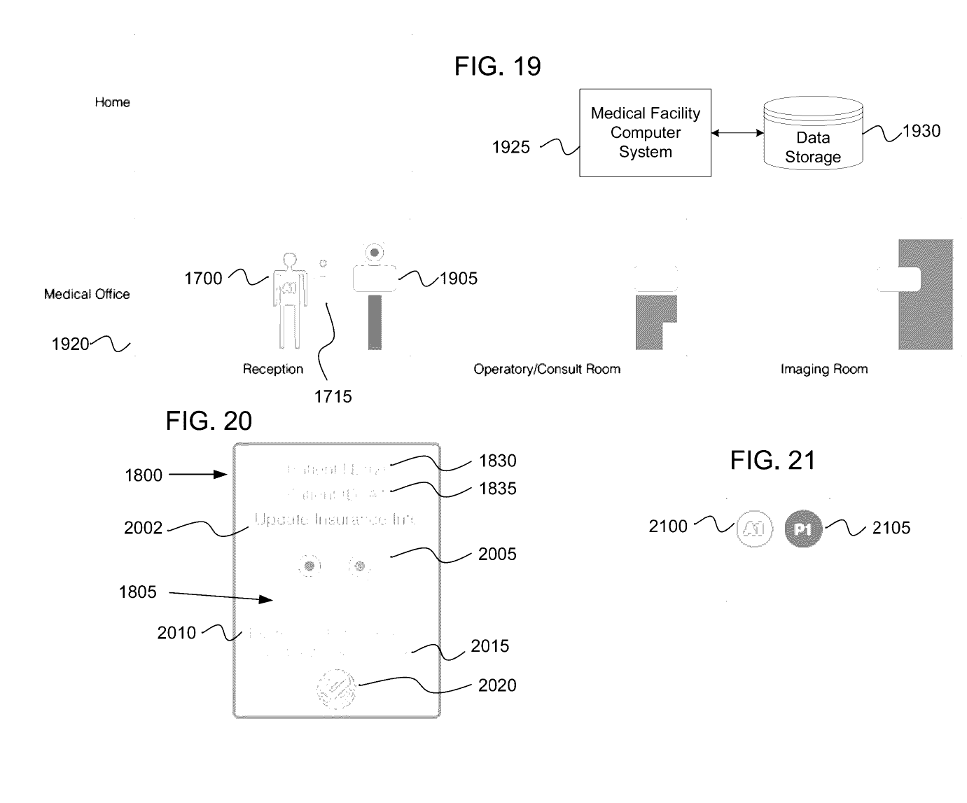

The patient 1700 arrives at the medical facility 1705 on his appointment date 1845, as shown in FIG. 19. On entering the reception area 1920, the mobile device 1715 with micro-location technology connects with micro-locations 1905 within the reception area 1920. The mobile device 1715 and the micro-locations 1905 communicate when the distance is less than a proximity threshold between the mobile device 1715 and the micro-locations 1905. Based on the unique patent ID 1835, the micro-locations 1905 detect the presence of the patient 1700. The micro-locations 1905 communicate with the medical facility computer system 1925 including data storage 1930 to confirm the appointment date 1845 and check-in the patient 1700. The micro-locations 1905 also send a check-in message to the mobile device 1715 confirming the check-in. Since the communication is automatically established when the mobile device 1715 comes within range of the micro-locations 1905, the patient 1700 does not need to manually check-in.

FIG. 20 illustrates the GUI 1805 of the patient pass 1800 after the patient pass 1800 receives the check-in message from the micro-locations 1905 via the mobile device 1715. As in previous embodiments, the patient pass 1800 allows the patient 1700 to update insurance information. The GUI 1805 includes an update insurance link 2002 that when selected, prompts the patient 1700 to enter new insurance information. The GUI 1805 displays a linked micro-location graphic 2005 that signifies that the micro-locations 1905 have detected the mobile device 1715. A micro-location detected message 2010 is then displayed by the GUI 1805. As in the previous examples, a patient checked-in message 2015 is displayed along with a checkmark 2020 indicating a successful check-in.

The patient pass 1800 with micro-location technology performs many of the same functions as the patient pass 200. For example, in FIG. 21, the patient pass 1800 links patient metadata 2100 and medical facility metadata 2105 on a successful check-in and stores the metadata in memory on the mobile device 1715 for later access.

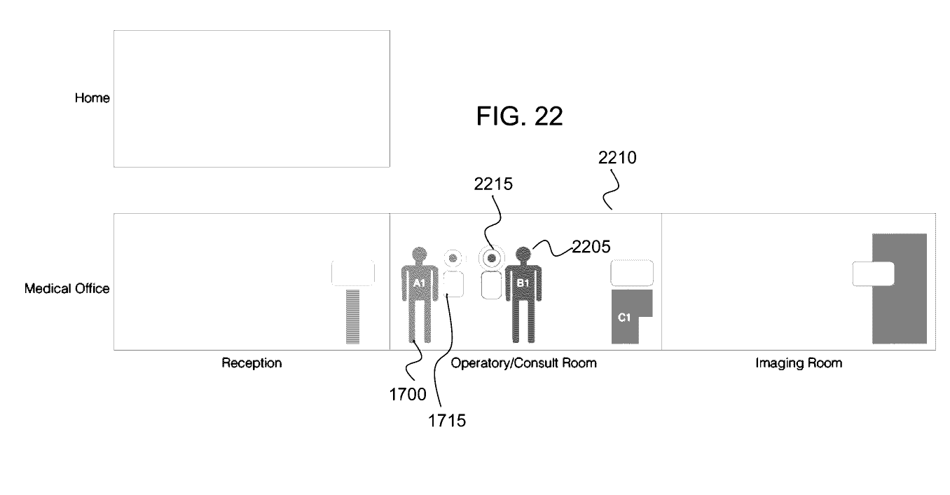

As illustrated in FIG. 22, when a medical professional 2205 is ready to see the patient 1700, the patient 1700 proceeds to a consultation room 2210. The medical professional 2205 brings a mobile device 2215, which includes micro-location technology. The medical professional 2205 logs in to the mobile device 2215 and accesses interface software on the mobile device 2215. The medical professional 2205 enables the micro-location technology on the mobile device 2215. Once enabled and once within a threshold range, the mobile device 2215 and the mobile device 1715 communicate. The medical professional 2205 can then access patient information on the mobile device 1715 of the patient 1700.

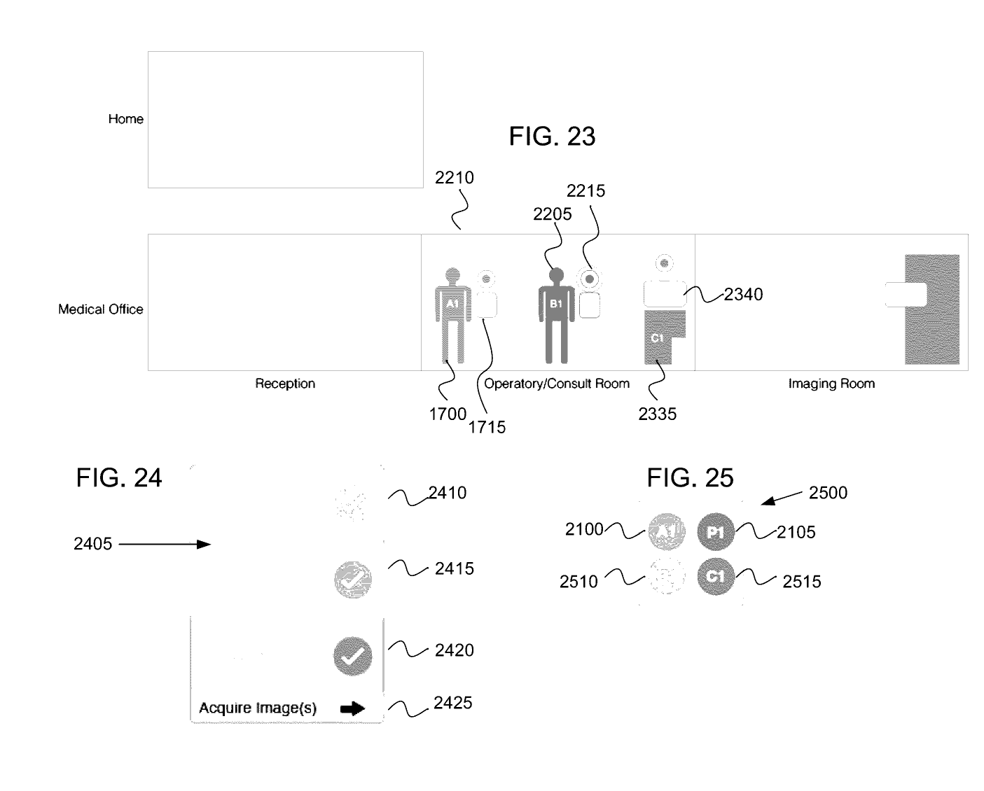

FIG. 23 illustrates a workflow where several micro-location devices communicate and share information about the patient 1700, the medical professional 2205, and the imaging device 2335. The mobile device 1715, the mobile device 2215, and a module 2340 detect the micro-location technology in each respective device and establish communication between the devices. The mobile device 1715 transmits information about the patient 1700 to the mobile device 2215 and the module 2340. Similarly, the mobile device 2215 transmits information about the medical professional 2205 to the module 2340 and the module 2340 transmits information about the imaging device 2335 to the mobile device 2215. The interface software can be loaded on the mobile device 2215 or on the module 2340 or both. In one embodiment, the medical professional does not require a mobile device 2215, but rather logs in to the module 2340 with unique identification information. The medical professional 2205 can access the interface software directly on the module 2240. As in the previous embodiments, the module 2340 communicates with the imaging device 2335 either by a wired or wireless connection.

FIG. 24 illustrates a GUI 2405 of the interface software. The GUI 2400 displays information that is received from the other devices. As in previous embodiments, the GUI 2405 displays the personnel name and personnel ID 2410, the patient name and patient identification number 2415, and the consultation room imaging device ID 2420. The medical professional 2205 can confirm that the information on the GUI 2405 is correct before acquiring diagnostic images via the acquire image link 2425. After meeting with the patient 1700, the medical professional 2205 takes diagnostic images of the patient 1700 using the imaging device 2235. The medical professional 2205 selects the acquire image link 2425 on the GUI 2405 to merge the diagnostic images with the information in the interface software.

Once the diagnostic images are acquired, the interface software links the diagnostic images with metadata 2500, as shown in FIG. 25. Metadata 2500 includes the patient metadata 2100, the medical facility metadata 2105, the medical professional metadata 2510, and the imaging device metadata 2515. This metadata 2500 is appended to the images acquired by the imaging device 2335. As in the previous embodiment, the metadata 2500 appended to the diagnostic images enables a subsequent user of the images to quickly determine the information contained in the metadata 2400.

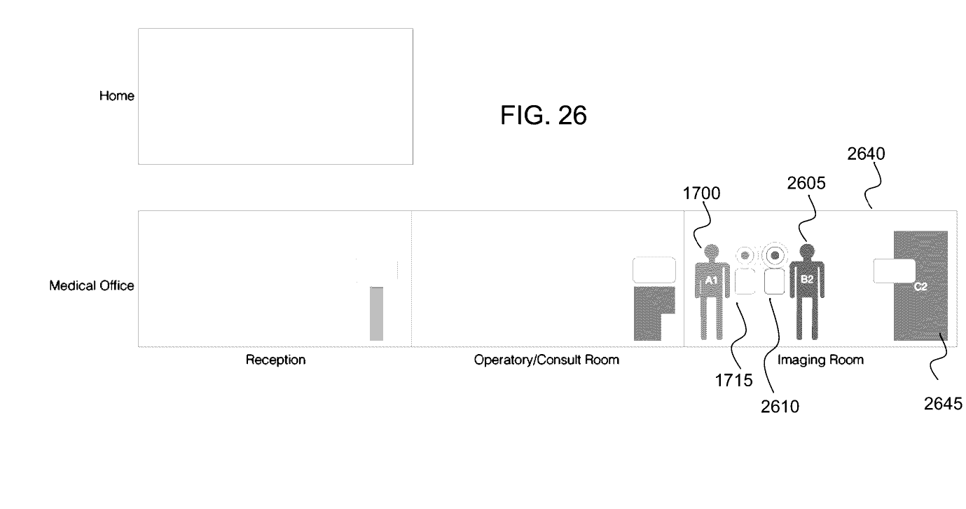

When the patient 1700 is finished with a portion of the medical service in the consultation room 2210, the patient 1700 may require additional diagnostic imaging from the imaging room 2640 as shown in FIG. 26. In this embodiment, a medical professional 2605 has a mobile device 2610, which is enabled with micro-location technology. The mobile device 2610 includes the interface software and automatically links to the mobile device 1715 through the micro-location technology. The mobile device 2600 receives information from the mobile device 1715 and the interface software stores the information in memory of the mobile device 2600. The interface software also transmits the information to the medical facility computer system 1925.

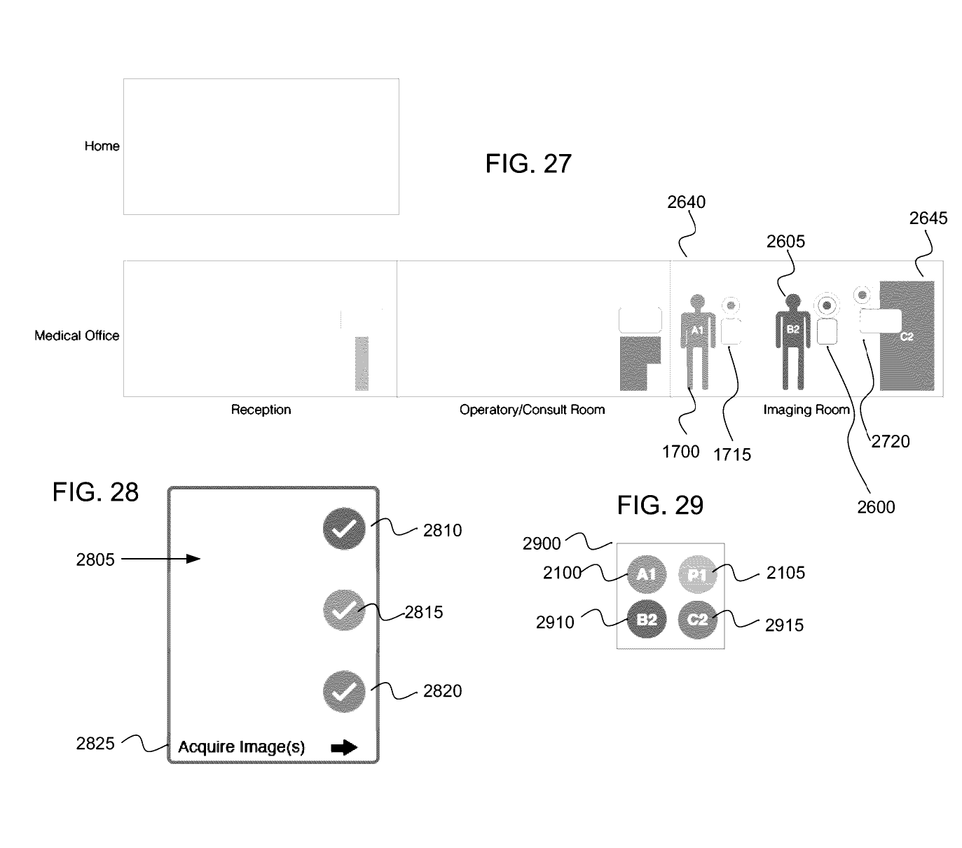

In the next step, as shown in FIG. 27, the mobile device 1715, the mobile device 2600, and the imaging device 2645 automatically detect each device through micro-location technology. Once the medical professional 2605 enables micro-location on the mobile device 2600, information is transferred from the mobile device 1715 and from a module 2720 to the mobile device 2600. As in the previous embodiments, the interface software can be located on the mobile device 2600 or on the module 2720 or both. If the interface software is located on the module 2720, the mobile device 2600 may not be required, since the medical professional 2605 may log-in to the interface software via the module 2720.

A GUI 2805 for the interface software is illustrated in FIG. 28. In this embodiment, the GUI 2805 includes medical professional information 2810, patient information 2815, and imaging device information 2820. As in the previous embodiments, the GUI 2900 displays this information so that the medical professional 2605 can determine if the information displayed on the GUI 2805 is correct. The GUI 2805 includes an acquire image link 2825 that tells the interface software to acquire diagnostic images from the imaging device 2645.

Metadata 2900 of the interface software is illustrated in FIG. 29. The metadata 2900 includes patient metadata 2100, medical facility metadata 2105, medical professional metadata 2910, and imaging device metadata 2915. The medical professional metadata 2910 is generated when the medical professional 2605 logs in to the interface software. The imaging device metadata 2915 can be acquired from the imaging device 2645 before or after acquiring diagnostic images. The metadata 2900 is appended to the diagnostic images taken with the imaging device 2645.

It should be noted that the methods described above are not limited to the specific examples presented. In particular, the workflows, the processes, and the steps can be performed in alternate orders. For example, the medical service that is performed in the imaging room 140 can be performed before the medical service in the consultation room 130. Also, obtaining information from the computer-readable, graphical codes or the micro-location devices can be performed in alternate orders, such as, for example, after the diagnostic images are captured. Additionally, the medical professionals and the medical mobile devices may be the same person or the same device, respectively. The medical professionals may include professionals of various occupations. For example, the medical professionals may be dentists, doctors, nurses, technicians, support staff, etc.

Thus, the invention provides, among other things, workflows and processes for performing medical services at a medical facility by using devices to help ensure accurate communication between the various parties and equipment. Various features and advantages of the invention are set forth in the following claims.

What is claimed is:

1. A method of using a computer-readable, graphical code on a mobile device to deliver a medical service, the method comprising:
generating a computer-readable, graphical code patient identifier and linking the identifier to appointment information and patient information in a data storage device;
sending the computer-readable, graphical code patient identifier and the appointment information to a mobile device from a medical facility computer system;
reading, by an optical scanner, the computer-readable, graphical code patient identifier at a medical facility; and
recognizing the patient at the medical facility when the computer-readable, graphical code patient identifier matches the appointment information and patient information for the patient stored in the data storage device.

2. The method of claim 1, wherein the computer-readable, graphical code patient identifier includes at least one selected from the group consisting of previously stored contact information and previously stored insurance information, and further comprising:
updating at least one selected from the group consisting of contact information and insurance information associated with the patient via a graphical user interface of the mobile device; and
updating the data storage device with at least one selected from the group consisting of the updated contact information and the updated insurance information after the computer-readable, graphical code patient identifier is read by the optical scanner.

3. The method of claim 1, further comprising:
sending a request to update at least one selected from the group consisting of contact information and insurance information associated with the patient;
updating the at least one of contact information and insurance information associated with the patient via a graphical user interface of a computer;
sending the at least one of updated contact information and updated insurance information associated with the patient to the data storage device; and
updating the data storage device with the at least one of contact information and insurance information associated with the patient.

4. The method of claim 1, wherein reading, by an optical scanner, the computer-readable, graphical code patient identifier at a medical facility includes doing so in a first room, and the method further comprises:
reading, by an optical scanner in a second room, the computer-readable, graphical code patient identifier;
acquiring an image by an imaging device in the second room; and
linking the image with patient information metadata associated with the computer-readable, graphical code patient identifier.

5. The method of claim 4, further comprising:
reading, by an optical scanner, a computer-readable, graphical code operator identifier for an operator acquiring images;
linking the image with operator information metadata associated with the computer-readable, graphical code operator identifier.

6. The method of claim 4, further comprising:
at least one selected from the group consisting of linking the image with medical facility information metadata associated with the optical scanner for admitting patients, linking the image with imaging device metadata associated with the imaging device, and linking the image with operator information metadata associated with an operator acquiring images.

7. The method of claim 1, further comprising:
reading, by a portable optical scanner, a barcode device identifier associated with an imaging device;
acquiring an image by the imaging device; and
linking the image with imaging device metadata associated with the barcode device identifier.

8. The method of claim 1, further comprising:
storing an image with metadata in data storage device, wherein the metadata includes at least one from the group consisting of patient information, operator information, medical facility information, and imaging device information.

9. The method of claim 1, wherein the computer-readable, graphical code patient identifier uniquely identifies the patient.

10. The method of claim 1, wherein the computer-readable, graphical code patient identifier is selected from the group consisting of a two-dimensional (2D) barcode, a matrix barcode, a quick response (QR) code, an Aztec code, a data matrix code, and a one-dimensional (1D) barcode.

11. A method of using an electronic micro-location patient identifier on a mobile device to deliver a medical service, the method comprising:
generating an electronic micro-location patient identifier and linking the identifier to appointment information for an appointment and patient information in a data storage device based on a request for a medical service;
sending the electronic micro-location patient identifier and the appointment information to a mobile device;
transmitting, by the mobile device to a micro-location device for checking-in patients, the electronic micro-location patient identifier at a medical facility when the mobile device comes within a first proximity threshold of distance from the micro-location device; and
checking-in the patient at the medical facility to perform the medical service during an appointment window when the electronic micro-location patient identifier matches the appointment information and patient information for the patient stored in the data storage device.

12. The method of claim 11, wherein the electronic micro-location patient identifier includes at least one selected from the group consisting of previously stored contact information and previously stored insurance information associated with the patient, and further comprising:
updating, via a graphical user interface of the mobile device, at least one selected from the group consisting of contact information and insurance information associated with the patient; and
updating the data storage device with the at least one selected from the group consisting of updated contact information and the updated insurance information after the electronic micro-location patient identifier is acquired by the micro-location device.

13. The method of claim 11, wherein transmitting, by the mobile device to a micro-location device for checking-in patients, the electronic micro-location patient identifier at a medical facility includes doing so in a first room, and the method further comprises:
transmitting, by the mobile device to a micro-location device in a second room, the electronic micro-location patient identifier when the mobile device comes within a second proximity threshold of distance from the micro-location device in the second room;
acquiring an image by an imaging device in the second room; and
linking the image with patient information metadata associated with the electronic micro-location patient identifier.

14. The method of claim 13, further comprising:
transmitting, by an operator mobile device to a micro-location device in the second room, an electronic micro-location operator identifier for an operator acquiring images;
linking the image with operator information metadata associated with the electronic micro-location operator identifier.

15. The method of claim 13, further comprising:
at least one selected from the group consisting of linking the image with medical facility information metadata associated with the micro-location device for checking-in patients, linking the image with imaging device metadata associated with the imaging device, or linking the image with operator information metadata associated with an operator acquiring images.

16. The method of claim 11, further comprising:
reading, by a portable micro-location device, a micro-location device identifier associated with an imaging device;
acquiring an image by the imaging device; and
linking the image with imaging device metadata associated with the micro-location device identifier.

17. The method of claim 11, further comprising:
storing an image with metadata in data storage device, wherein the metadata includes patient information, operator information, medical facility information, or imaging device information.

18. The method of claim 11, wherein the micro-location device is included in at least one from the group consisting of a second mobile device and a stationary computer.

19. A system configured to use an electronic patient identifier on a mobile device to facilitate the delivery of medical services, the system comprising:
a mobile device to receive an electronic patient identifier associated with a patient;
a computer configured to
generate patient information and appointment information for a medical service for the patient,
generate a computer-readable, graphical code patient identifier,
link the identifier to the appointment information and the patient information,
send the computer-readable, graphical code patient identifier and the appointment information to the mobile device, and
generate a checked-in notification;
a scanner configured to read the electronic patient identifier; and
a data storage device to store the appointment information, the patient information, and the electronic patient identifier associated with the patient.

20. The system of claim 19, wherein the electronic patient identifier includes a barcode displayed by the mobile device, the barcode selected from the group consisting of a two-dimensional (2D) barcode, a matrix barcode, a quick response (QR) code, an Aztec code, a data matrix code, and a one-dimensional (1D) barcode.

21. The system of claim 19, wherein the scanner is included in at least one selected from the group consisting of a second mobile device or a stationary computer.

22. The system of claim 19, wherein the mobile device is further configured to update at least one selected from the group consisting of contact information and insurance information associated with the patient via a graphical user interface of the mobile device, and the computer is configured to update the at least one of the contact information and the insurance information embedded in the electronic patient identifier.

23. The system of claim 19, further comprising:
an imaging device configured to acquire an image of the patient;
a second scanner configured to read the electronic patient identifier, wherein the second scanner is associated with at least one selected from the group consisting of the imaging device, a patient room, and an imaging room;
wherein the computer is further configured to link the image with patient information metadata associated with the electronic patient identifier.

24. The system of claim 23,
wherein the second scanner is further configured to read an electronic operator identifier for an operator acquiring images, and the computer is further configured to link the image with operator information metadata associated with the electronic operator identifier; or
wherein the computer is further configured to link the image with medical facility information metadata associated with the scanner; or
wherein the computer is further configured to link the image with imaging device metadata associated with the imaging device; or
wherein the second scanner comprises a portable scanner configured to read a device identifier associated with the imaging device, and the computer is further configured to link the image with the imaging device metadata associated with the imaging device.

26. The system of claim 24, wherein the data storage device is further configured to store the image with metadata in data storage device, wherein the metadata includes patient information, operator information, medical facility information, or imaging device information.
